(19) United States
(12) Patent Application Publication
(10) Pub. No.: US 2024/0339215 A1
(43) Pub. Date: Oct. 10, 2024

(54) SYSTEM AND METHOD FOR DRUG SELECTION

(71) Applicant: Board of Regents, The University of Texas System, Austin, TX (US)

(72) Inventors: Song Stephen Yi, West University Place, TX (US); Daniel Camilo Osorio Hurtado, Pflugerville, TX (US); Willard Bui, Austin, TX (US); Nidhi Sahni, Austin, TX (US)

(21) Appl. No.: 18/431,060

(22) Filed: Feb. 2, 2024

Related U.S. Application Data
(60) Provisional application No. 63/494,544, filed on Apr. 6, 2023.

(51) Int. Cl. G16H 50/20; G16B 20/00; G16B 40/20

(52) U.S. Cl. CPC G16H 50/20; G16B 20/00; G16B 40/20

(57) ABSTRACT

A method of treating a subject for a disease comprises obtaining a first data set of cell transcriptomic data, obtaining a second set of cell transcriptomic data, obtaining a set of effectiveness data related to a plurality of candidate medications or treatments related to the samples, calculating a lower-dimensional model from the first set of cell transcriptomic data, assigning the second set of cell transcriptomic data to at least one group of the plurality of groups, calculating a synergistic effectiveness of combinations in a set of candidate medication or treatment combinations related to each group of the plurality of groups of cell lines, selecting a treatment combination from the set of candidate medication or treatment combinations whose synergistic effectiveness is above a threshold for the at least one group, and treating the subject with the selected treatment combination.

### CROSS-REFERENCE TO RELATED APPLICATIONS

This application claims priority to U.S. Provisional Patent Application No. 63/494,544, filed on Apr. 6, 2023, incorporated herein by reference in its entirety.

### STATEMENT REGARDING FEDERALLY SPONSORED RESEARCH OR DEVELOPMENT

This invention was made with government support under Grant no. R35 GM133658 awarded by the National Institutes of Health. The government has certain rights in the invention.

### BACKGROUND OF THE INVENTION

Cancer is a significant public health problem; it is currently the second leading cause of death for men and women in the US, passed only by heart disease. Aware of the limitations associated with the adoption of single-cell RNA-seq characterization of patients in clinical practice, the performance of various approaches for recapitulation of the high-resolution single-cell RNA-seq data was assessed, with the goal of leveraging molecular profiles from large cohorts already available, such as the TCGA project. Deconvolution of bulk RNA-seq profiles from a large cohort of patients into cell lines provides unbiased knowledge for resource prioritization in cancer research with a high impact on public health. Deconvolution methods have demonstrated promising results in classifying heterogeneous tissues into distinct cell types. However, their utility in identifying sample fractions using homogeneous cell types with minor molecular phenotype changes as reference has not been established.

Precision oncology seeks to match patients to the optimal pharmacological regimen; yet, due to tumor heterogeneity, this is challenging. Numerous studies have been conducted to produce clinically relevant pharmacological response forecasts by integrating modern machine learning algorithms and several data types. Insufficient patient numbers and lack of knowledge of the molecular targets for each drug under study limit their use. The present disclosure uses single-cell RNA-seq based transfer learning to contextualize patients' tumor cells in terms of their more similar cell lines with known susceptibility to drug combinations. The disclosed system and methods maximize the translational potential of in-vitro assays for identifying synergistic drug combinations and prioritizing them for clinical use. Consistent findings in a cohort of breast cancer patients corroborated the understanding of the disease's molecular subtypes. To aid in creating personalized treatments and data-driven clinical trials, the most prevalent cell lines were identified and synergistic combinations were prioritized based on tumor compositions at various resolution levels.

Precision oncology strives to match patients with the best pharmaceutical regimen for treating their cancer. Due to the heterogeneity of malignancies, selecting the right pharmacological regimen is a difficult challenge. Tumors are made up of a variety of cell types that interact and modify the extracellular matrix, influencing the immune system's ability to recognize and kill malignant cells. (See FIG. 1)

For that reason, cancer patients are usually categorized into subgroups based on histological or molecular characteristics such as cell morphologies, DNA mutations, or gene expression patterns to improve treatment efficacy (Rouzier, et al., Clinical cancer research, 2005; and Prat, et al., The Breast, 2015). Using this approach, only patients having characteristics that are comparable to those of others who have had a positive response to treatment are given the medicine (Zhou, et al., The lancet oncology, 2011). This approach is advantageous because, while some patients benefit from standard therapies, many patients suffer from acquired resistance to therapy, which can cause their tumors to relapse over time.

Several research groups have recently attempted to incorporate multiple sources of information into cutting-edge machine learning approaches in order to create clinically oriented predictions of patient responses to medications. I-PREDICT, NCI-MATCH, MI-ONCOSEQ, WINTHER, ONCOTARGET/ONCOTREAT, SELECT, and ENLIGHT are studies that aim to match patients with therapies and predict their impact on treatment outcome using DNA biomarkers, genomic and transcriptomic information, protein-protein interaction networks, synthetic lethal and/or synthetic rescue interactions among other factors. However, developing such studies requires data from a large number of patients as well as extensive knowledge of the exact molecular targets for each of the drugs under investigation, limiting their applicability.

In-vitro cell line assays, on the other hand, are still the first line of cancer research since they serve as surrogates for the patient's tumor response. Cancer cell lines are a low-cost, easy-to-maintain model system for medical research suitable to be manipulated to represent the genetic aberrations found in patients' tumors. When integrated into panels, cell lines represent the genetic diversity and phenotypic variability of the patients; hence, it is necessary to develop strategies to optimize the translational potential of in-vitro cellular responses into pharmaceutical alternatives for patients without the need of a reference group.

With the introduction of new high-throughput experimental approaches and computational methods that allow for the characterization of the transcriptional profile at the single-cell level, it is now possible to identify isogenic sub-populations of cells responding differently to the drug under study (Navin, Genome research, 2015). When applied to tumors, single cell approaches allow for the identification of all the proportions of cell types making part of it (Zilionis, et al., Immunity, 2019), and when both sources of information (responses of purified cell lines and tumor data) are combined, single-cell transcriptomics offers the ability to maximize the translational potential of in-vitro cellular responses into patients' pharmacological options.

Recently, Gambardella and collaborators released a single-cell atlas of breast cancer cell lines Gambardella, et al., Nature Communications, 2022. The atlas reports single-cell RNA-seq data for 35,276 individual cells from 32 breast cancer cell lines individualized using DROP-seq (Macosko, et al., Cell, 2015). Concurrently, using 10× Chromium, Wu and collaborators published a single-cell atlas of human breast cancers that includes 130,246 annotated single cells from 26 primary tumors, including 11 ER⁺, 5 HER2⁺ and 10 TNBCs (Wu, et al., Nature genetics, 2021). Together, both data sets provide an opportunity to contextualize the cancer cells from patients' tumors in terms of cancer cell lines using transfer learning.

Transfer learning is a machine learning technique that uses a joint embedding to map query data sets on top of a given reference. This method allows for integration of datasets after removing non-biological variation and for contextualizing new data sets using the metadata associated with the existing reference with high precision and without the need for recomputing the reference embedding.

The ability to contextualize tumor cells in terms of more similar cell lines opens up a world of possibilities for mining hundreds of publicly-available cell line assays to identify appropriate pharmaceutical options for treating patients in a tailored manner without using a reference cohort or having a deep understanding of the drugs' molecular targets. Additionally, this strategy may be broadly used in studying any illness for which a sufficient number of cell lines representing patients' genetic and phenotypic heterogeneity as well as in-vitro assays are available.

The disclosed systems and methods use the systematic evaluation of 1,275 pairwise drug combinations tested over 51 breast cancer cell lines using the Genomics of Drug Sensitivity in Cancer (GDSC) cell line screening platform to prioritize synergistic drug combinations based on patients' tumor composition, aiming to improve patients' survival rate through personalized medicine (see Jaaks, et al., Nature, 2022). The data was also examined at the population level to calculate which cell lines and drug combinations to prioritize for drug-development and clinical trial purposes in each breast cancer molecular subtype. Tumor cell transcriptional profiles were able to be traced to their most similar cell lines using a transfer learning data-driven computational approach as a compass. The disclosed experimental examples indicate that transfer learning is valuable for precision medicine because it bridges the gap between in-vitro drug sensitivities and pharmacological treatment options for a given patient or patient subgroup.

### SUMMARY OF THE INVENTION

In one aspect, a method of treating a subject for a disease comprises obtaining a first data set of cell transcriptomic data related to samples of cell lines taken from subjects diagnosed with the disease, obtaining a second set of cell transcriptomic data related to samples of cell lines taken from a subject to be treated, obtaining a set of effectiveness data related to a plurality of candidate medications or treatments related to the samples, calculating a lower-dimensional model from the first set of cell transcriptomic data in order to group the cell lines into a plurality of groups, assigning the second set of cell transcriptomic data to at least one group of the plurality of groups, calculating a set of candidate medication or treatment combinations from the plurality of candidate medications or treatments, each combination in the set comprising N treatments or medications, where N>1, calculating a synergistic effectiveness of each combination in the set of candidate medication or treatment combinations related to each group of the plurality of groups of cell lines, selecting a treatment combination from the set of candidate medication or treatment combinations whose synergistic effectiveness is above a threshold for the at least one group, and treating the subject with the selected treatment combination.

In one embodiment, the step of calculating the lower-dimensional model from the first set of cell transcriptomic data comprises the step of discarding one or more dimensions or parameters of the data. In one embodiment, the step of calculating the lower-dimensional model from the first set of cell transcriptomic data comprises the step of combining two or more dimensions or parameters of the data. In one embodiment, the step of calculating the lower-dimensional model from the first set of cell transcriptomic data comprises the use of a dimensionality reduction technique selected from principal component analysis and grouping high-dimensional data into clusters. In one embodiment, the step of calculating the lower-dimensional model from the first set of cell transcriptomic data comprises assigning weights to one or more parameters of the first set of cell transcriptomic data and discarding parameters weighted below a threshold.

In one embodiment, the step of calculating the lower-dimensional model from the first set of cell transcriptomic data comprises assigning weights to one or more parameters of the first set of cell transcriptomic data and selecting the N highest weighted parameters. In one embodiment, the step of calculating the lower-dimensional model from the first set of cell transcriptomic data comprises using k-means clustering. In one embodiment, the step of assigning the second set of cell transcriptomic data to at least one group of the plurality of groups comprises calculating the k-nearest neighbor of each element of the second set of cell transcriptomic data. In one embodiment, the disease is cancer and the first set of cell transcriptomic data comprises cells selected from cancer cell lines, tumor cell lines, breast cancer cell lines, pancreatic cancer cell lines, or other tumor cell lines.

In one aspect, a system for selecting a treatment combination for a subject comprises a non-transitory computer-readable medium with instructions stored thereon, which when executed by a processor, perform steps comprising obtaining a first data set of cell transcriptomic data related to samples of cell lines taken from subjects diagnosed with the disease, obtaining a second set of cell transcriptomic data related to samples of cell lines taken from a subject to be treated, obtaining a set of effectiveness data related to a plurality of candidate medications or treatments related to the samples, calculating a lower-dimensional model from the first set of cell transcriptomic data in order to group the cell lines into a plurality of groups, assigning the second set of cell transcriptomic data to at least one group of the plurality of groups, calculating a set of candidate medication or treatment combinations from the plurality of candidate medications or treatments, each combination in the set comprising N treatments or medications, where N>1, calculating a synergistic effectiveness of each combination in the set of candidate medication or treatment combinations related to each group of the plurality of groups of cell lines, and selecting a treatment combination from the set of candidate medication or treatment combinations whose synergistic effectiveness is above a threshold for the at least one group.

In one embodiment, the step of calculating the lower-dimensional model from the first set of cell transcriptomic data comprises the step of discarding one or more dimensions or parameters of the data. In one embodiment, the step of calculating the lower-dimensional model from the first set of cell transcriptomic data comprises the step of combining two or more dimensions or parameters of the data. In one embodiment, the step of calculating the lower-dimensional model from the first set of cell transcriptomic data comprises the use of a dimensionality reduction technique selected from principal component analysis and grouping high-dimensional data into clusters. In one embodiment, the step of calculating the lower-dimensional model from the first set of cell transcriptomic data comprises assigning weights to one or more parameters of the first set of cell transcriptomic data and discarding parameters weighted below a threshold.

In one embodiment, the step of calculating the lower-dimensional model from the first set of cell transcriptomic data comprises assigning weights to one or more parameters of the first set of cell transcriptomic data and selecting the N highest weighted parameters. In one embodiment, the step of calculating the lower-dimensional model from the first set of cell transcriptomic data comprises using k-means clustering. In one embodiment, the step of assigning the second set of cell transcriptomic data to at least one group of the plurality of groups comprises calculating the k-nearest neighbor of each element of the second set of cell transcriptomic data. In one embodiment, the disease is cancer and the first set of cell transcriptomic data comprises cells selected from cancer cell lines, tumor cell lines, breast cancer cell lines, pancreatic cancer cell lines, or other tumor cell lines.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a diagram of a method of the disclosure compared to current methods.

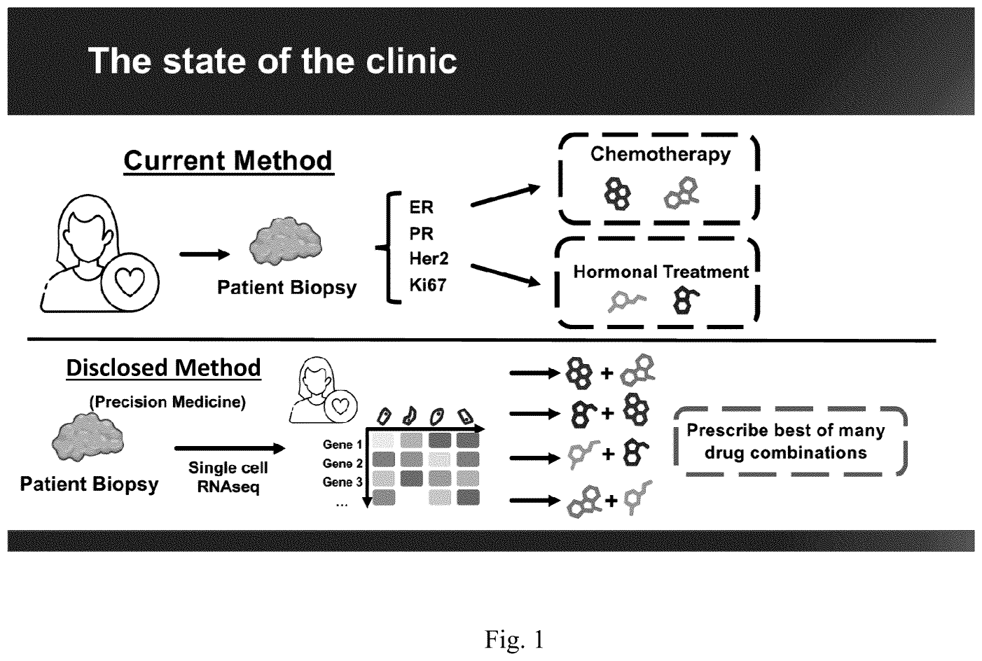

FIG. 2 is an exemplary computing device.

FIG. 3 is a graphical depiction of a method of the disclosure.

FIG. 4A is a Uniform Manifold Approximation and Projection (UMAP) displaying the harmonized transcriptional profile of 32 breast cancer cell lines sequenced using DROP-seq.

FIG. 4B is a graph showing accuracy evaluation using cross-validation. The percentage of cells utilized for training is displayed, as well as the accuracy and 95% confidence intervals computed using the binomial test. Accuracy is defined as the percentage of correctly classified observations divided by the total number of observations.

FIG. 4C shows an accuracy evaluation using wild-type cells from a different single-cell technology. Transcriptomes of cells derived from the wild-type MCF7 cell line that were sequenced using 10× Chromium were appropriately labelled in 97.9% of cases.

FIG. 4D shows an accuracy evaluation using cells from a different single-cell technology carrying a genetic modification. Transcriptomes of cells originating from the T47D cell line carrying the CDH1 gene knockout through CRISPR and sequenced using 10× Chromium were labelled correctly in all cases.

FIG. 4E shows an accuracy evaluation using cells from a different single-cell technology under cancer treatment. Transcriptomes of cells obtained from the BT474 cell line that were treated with 1 μM of Lapatinib for 10 days and sequenced using 10× Chromium were identified correctly in 93.1% of instances.

FIG. 5A shows transcriptional profiles of a patient's tumor cells mapped to their more comparable transcriptional profile within the integrated reference population of breast cancer cell lines.

FIG. 5B shows validation of the accuracy of the mapping procedure by leave-one-out cross validation (LOOCV). The CID44971 donor's cancer cells were used, and the average and standard error of the relative frequency for each cell line are reported in percentages.

FIG. 5C shows the composition of cancer tumor cells in terms of cell lines for twenty patients described in the breast cancer cell atlas.

FIG. 6A shows a determination of patient's tumor composition. The composition of malignant tumor cells, expressed as a proportion (rounded to two decimals) of cell lines, for twenty patients described in the breast cancer cell atlas.

FIG. 6B shows an identification of the tumor compositions of a population of patients. The composition in percentages of cancer tumor cells in terms of cell lines for twenty patients reported in the breast cancer cell atlas is summarized by molecular subtypes.

FIG. 7 shows an evaluation of the precision of CIBERSORTx's deconvolution proportions.

FIG. 8 shows a hierarchical clustering of computed pseudo-bulk profiles using all cells. Patient profiles (CID*) were generated using all cells reported in the atlas. The percentage of bootstraps (bp) in which the relationship is recovered is indicated in green.

FIG. 9 shows a hierarchical clustering of computed pseudo-bulk profiles using only cancer cells. Patient profiles (CID*) were generated using only the cancer cells reported in the atlas. The percentage of bootstraps (bp) in which the relationship is recovered is indicated in green.

FIG. 10A, FIG. 10B, and FIG. 10C show the prioritization of drug combinations exhibiting synergistic effects using uniform proportions of cell lines. FIG. 10A is computed for ER-positive cell lines. FIG. 10B is computed for HER2-positive cell lines. FIG. 10C is computed for TNBC cell lines.

FIG. 11A-FIG. 11F are graphs showing the prioritization of drug combinations exhibiting synergistic effects based on the tumor composition in terms of cell lines. FIG. 11A is computed for the CID44971 donor. FIG. 11B is computed for all donors included in the breast cancer atlas.

FIG. 11C is computed for donors with ER-positive malignancies. FIG. 11D is computed for donors with HER2-positive malignancies. FIG. 11E is computed for donors with TNBC malignancies. FIG. 11F is a comparison of the computed activity score across breast cancer sub-types.

FIG. 12 shows a distribution of the synergistic effect of Navoticlax combinations across breast cancer cell lines.

FIG. 13A shows an enrichment score for ER+ cell lines.

FIG. 13B shows an enrichment score for HER2+ cell lines.

FIG. 13C shows an enrichment score for TNBC cell lines.

FIG. 14 is a diagram of groups of calculated tumor composition.

FIG. 15A is a diagram showing a drug mapping to a diagram of groups of calculated tumor composition.

FIG. 15B is a set of pie charts showing calculated drug combinations for two exemplary patients.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

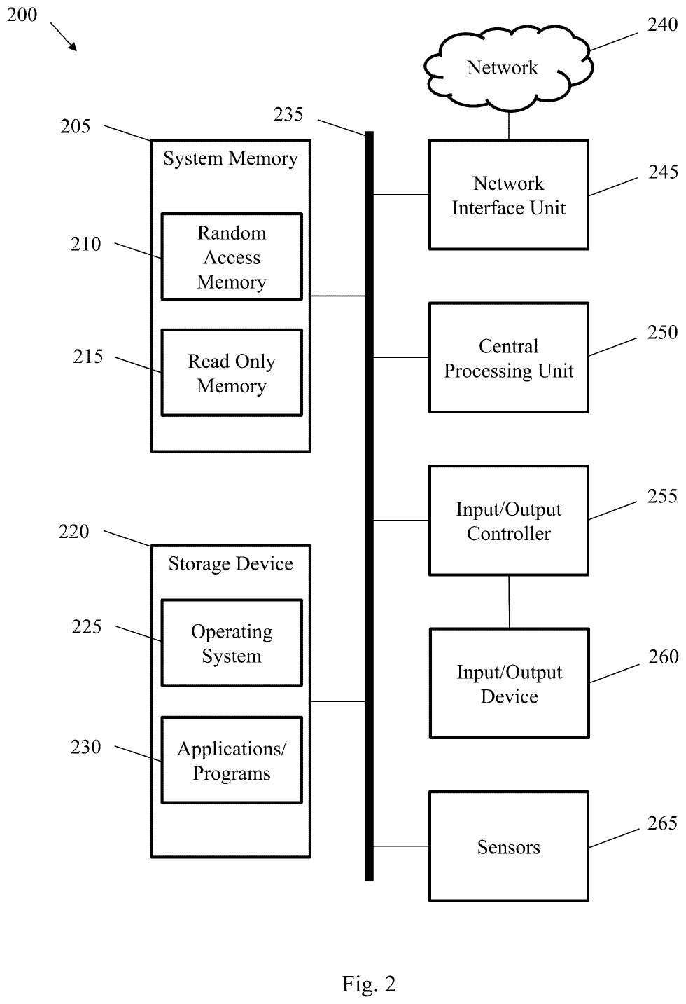

FIG. 2 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 2 depicts an illustrative computer architecture for a computer 200 for practicing the various embodiments of the invention. The computer architecture shown in FIG. 2 illustrates a conventional personal computer, including a central processing unit 250 (“CPU”), a system memory 205, including a random access memory 210 (“RAM”) and a read-only memory (“ROM”) 215, and a system bus 235 that couples the system memory 205 to the CPU 250. A basic input/output system containing the basic routines that help to transfer information between elements within the computer, such as during startup, is stored in the ROM 215. The computer 200 further includes a storage device 220 for storing an operating system 225, application/program 230, and data.

The storage device 220 is connected to the CPU 250 through a storage controller (not shown) connected to the bus 235. The storage device 220 and its associated computer-readable media provide non-volatile storage for the computer 200. Although the description of computer-readable media contained herein refers to a storage device, such as a hard disk or CD-ROM drive, it should be appreciated by those skilled in the art that computer-readable media can be any available media that can be accessed by the computer 200.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 200 may operate in a networked environment using logical connections to remote computers through a network 240, such as TCP/IP network such as the Internet or an intranet. The computer 200 may connect to the network 240 through a network interface unit 245 connected to the bus 235. It should be appreciated that the network interface unit 245 may also be utilized to connect to other types of networks and remote computer systems.

The computer 200 may also include an input/output controller 255 for receiving and processing input from a number of input/output devices 260, including a keyboard, a mouse, a touchscreen, a camera, a microphone, a controller, a joystick, or other type of input device. Similarly, the input/output controller 255 may provide output to a display screen, a printer, a speaker, or other type of output device. The computer 200 can connect to the input/output device 260 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 220 and/or RAM 210 of the computer 200, including an operating system 225 suitable for controlling the operation of a networked computer. The storage device 220 and RAM 210 may also store one or more applications/programs 230. In particular, the storage device 220 and RAM 210 may store an application/program 230 for providing a variety of functionalities to a user. For instance, the application/program 230 may comprise many types of programs such as a word processing application, a spreadsheet application, a desktop publishing application, a database application, a gaming application, internet browsing application, electronic mail application, messaging application, and the like. According to an embodiment of the present invention, the application/program 230 comprises a multiple functionality software application for providing word processing functionality, slide presentation functionality, spreadsheet functionality, database functionality and the like.

The computer 200 in some embodiments can include a variety of sensors 265 for monitoring the environment surrounding and the environment internal to the computer 200. These sensors 265 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

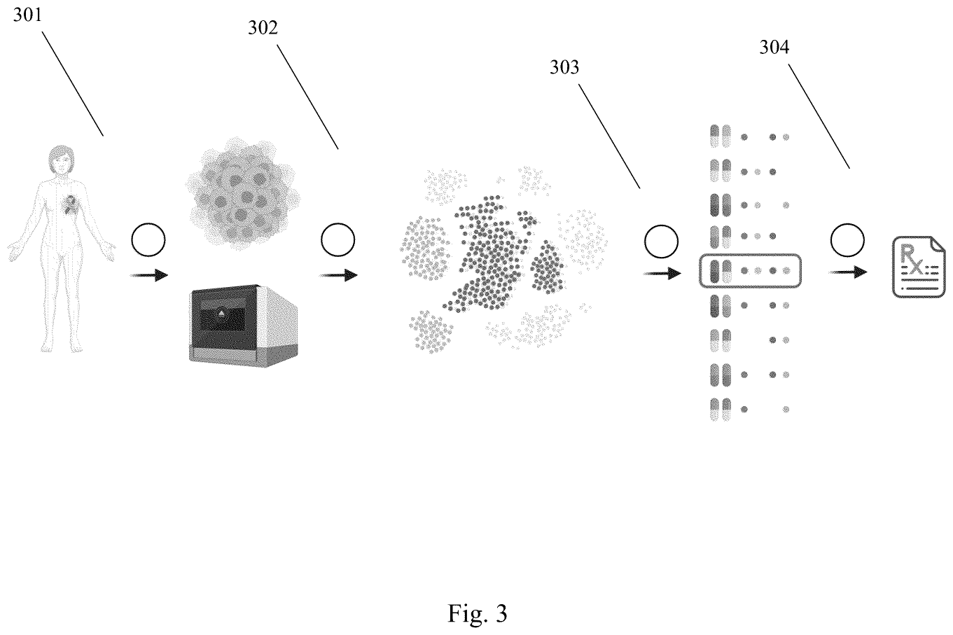

Overview of the drug combination prioritization procedure—The disclosed method for optimizing the translational potential of in-vitro tests for discovering synergistic drug combinations and selecting them for clinical application is divided into four general steps as described in FIG. 3

With reference to FIG. 3, in step 301, a biopsy from the patient tumor is collected and single-cell transcriptomes are sequenced using drop-based single-cell RNA-seq techniques. In step 302, using transfer learning, cells are mapped to an atlas of cancer cell lines derived from the tumor of origin and representing the phenotypic diversity of the malignancies affecting patients. In step 303, based on the detected tumor composition in terms of cell lines, the drug combinations with highest synergistic activity across the detected cell lines are identified and then in step 304, suggested for prescription to the patient.

The first step 301 is to sequence the transcriptome of cancerous tumor cells obtained from patients with the disease. Although certain examples and embodiments disclosed herein may be related to one or more different types of cancer, for example breast cancer, it is understood that the methods and systems disclosed herein may be applied to any cancer, including but not limited to breast cancer, lung cancer, head and neck cancer, bladder cancer, stomach cancer, cancer of the nervous system, bone cancer, bone marrow cancer, brain cancer, colon cancer, esophageal cancer, endometrial cancer, gastrointestinal cancer, genital-urinary cancer, stomach cancer, lymphomas, melanoma, glioma, bladder cancer, pancreatic cancer, gum cancer, kidney cancer, retinal cancer, liver cancer, nasopharynx cancer, ovarian cancer, oral cancers, bladder cancer, hematological neoplasms, follicular lymphoma, cervical cancer, multiple myeloma, osteosarcomas, thyroid cancer, prostate cancer, colon cancer, prostate cancer, skin cancer, stomach cancer, testis cancer, tongue cancer, or uterine cancer. In some embodiments, the cancer is a pre-cancer.

The second step 302 is to map the patient's cells to a more closely related cell line using a cell line atlas designed to represent the disease and contextualize tumor cells in terms of cell lines. In the third step, 303, the transfer learning process from step 302 is leveraged to close the gap between in-vitro drug sensitivities and pharmacological treatment options for the patient. This step assigns a ranking to the drug combinations based on their effect on the cell lines identified previously. Finally, in step 304, drug combinations that are expected to be effective in treating the patient are recommended for their prescription.

The ability to contextualize patients' tumor cells in terms of more similar cell lines opens up a world of possibilities for mining hundreds of publicly available cell line assays to identify appropriate pharmaceutical options for treating patients in a personalized manner without the need for a reference cohort or a thorough understanding of the drugs' molecular targets. To determine the best pharmacological regimen for each patient based on the cell line composition of their tumor, a collection of phenotypic responses (for example, survival) to drugs across cell lines is all that is required. However, because monotherapy in cancer is highly susceptible to resistance development after an initial response to treatment, combination therapy has become the standard pharmacological regimen for treating complex diseases like cancer. Combination therapy improves patient survival by halting tumor progression and preventing the development of drug resistance in cancer (see Chatterjee et al., Trends Cancer, 2019). As a result, the present disclosure focuses on identifying drug combinations with synergistic effects and ranking them based on the tumor composition of the patients.

In various embodiments, the present disclosure relates to systems and methods of using artificial intelligence (AI) or machine learning (ML) algorithms to treat patients, for example cancer patients, by selecting medications or medication combinations based on data related to tumor cells. In some embodiments, the disclosed systems and methods use transfer learning in order to categorize patients and/or tumors into groups based on morphological, genomic, and/or transcriptomic similarities in order to determine which medications or medication combinations are likely to be effective.

In some embodiments, the disclosed systems and methods may include steps of training an AI or ML algorithm by obtaining, labeling, and/or inputting data into a model in order to construct a reference data atlas comprising low-dimensional transcriptional profiles of a plurality of cell lines, for example cancer cell lines, tumor cell lines, breast cancer tumor cell lines, pancreatic cancer tumor cell lines, or tumor cell lines of any suitable cancer. The process of embedding involves the computation of a lower-dimensional representation from a high-dimensional representation of individual elements of a dataset, for example by discarding dimensions or parameters of the dataset which are irrelevant or not relevant above a given threshold to the parameter or parameters being measured, or by consolidating/compressing correlated or dependent dimensions or parameters of the dataset. The disclosed method may assign weights to various dimensions or parameters of the high-dimensional dataset. In some embodiments, the disclosed method comprises the step of selecting all assigned weights higher than a given threshold.

In some embodiments, the disclosed method comprises the step of selecting a set of the N highest weights in the assigned weights in order to identify the most relevant dimensions or parameters of the high-dimensional dataset.

In some embodiments, a system or method disclosed herein may comprise a dimensionality reduction technique, for example Principal Component Analysis (PCA). PCA is a dimensionality reduction technique that is often used to preprocess data before applying machine learning (ML) algorithms. The number of PCA components considered can have a significant impact on the performance of ML algorithms. When fewer PCA components are considered, the resulting data will have lower dimensionality, which can make it easier for ML algorithms to process. However, reducing the number of PCA components too much can lead to information loss, which can negatively affect the accuracy of the ML model. On the other hand, considering more PCA components can capture more of the variance in the data, which can help to improve the accuracy of the ML model. However, it can also increase the dimensionality of the data, making it more complex and potentially more difficult to process. This can lead to overfitting and decreased interpretability of the results. Thus, the number of PCA components to consider in any given implementation depends on various factors such as the complexity of the dataset, the desired level of accuracy, and the computational resources available.

In some embodiments, a method may include use of a grouping of high-dimensional data into k clusters. In some embodiments, k is representative of the number of clusters that the algorithm should group the data into. In such embodiments, when the value of k is increased, the algorithm will attempt to group the data into more clusters, which can lead to a more fine-grained clustering of the data. Increasing k too much can result in overfitting, where the algorithm creates too many clusters, each with only a few data points. This can result in a loss of generalization and increased complexity. Conversely, decreasing the value of k results in fewer clusters and a more coarse-grained clustering of the data. This can be useful when dealing with large datasets or when there is a prior belief that there are only a small number of distinct clusters in the data.

In some embodiments, the method may comprise a validation step, for example a cross-validation step, wherein a subset of a dataset with known output values is used to create a lower-dimensional model, and the created model is checked against a second subset of the dataset, some or all of which was not used in the creation of the lower-dimensional model. The performance of the model may then be assessed based on the accuracy of the results returned when applying the lower-dimensional model to the second subset of data. In some embodiments, where the cross-validation is successful, for example when the performance of the lower-dimensional model is above a certain accuracy threshold, the lower-dimensional model may then be considered verified. In some embodiments, a method may require performance of a certain number of cross-validation iterations, for example 1, 2, 3, 4, 5, 6, 7, 8, 9, or 10 iterations against mutually exclusive subsets of a dataset, before the lower-dimensional model is considered verified. In some embodiments, if a lower-dimensional model is considered to be below a given accuracy threshold, the lower-dimensional model may be discarded or re-formed incorporating additional data from the original dataset and/or other datasets.

Taking as an example a dataset with 1000 samples being used to train a model to make predictions on new, unseen data. One could randomly split the data into two sets: a training set with 800 samples and a test set with 200 samples. The training set could then be used to train the model, and the test set could then be used to evaluate its performance. However, there is a risk that the 200 samples in the test set are not representative of the entire dataset, and that the performance of the model on this particular test set is not indicative of its generalization performance. Cross-validation mitigates this risk by splitting the dataset into several subsets, or “folds.” For example, one could split the 1000-sample dataset into 5 folds of 200 samples each. One would then train the model 5 times, each time using a different fold as the test set and the remaining 4 folds as the training set. This yields five different performance metrics (one for each fold), which can then be averaged to get a more reliable estimate of the model's generalization performance. This in turn helps achieve a better understanding of how the model will perform on new, unseen data.

In some embodiments, a method as disclosed herein may further comprise calculating or obtaining an effectiveness score of one or more medications or treatments against one or more cell lines. In some embodiments, an effectiveness score may be calculated for a combination of two drugs, for example using the average of the synergistic effect of both drugs across different concentrations multiplied by the proportion of the cell line in tumor's composition.

In some embodiments, a system or method disclosed herein may comprise the step of computing an activity score of a two-, three-, four-, five-, or higher order combination of medications and/or treatments based on individual effectiveness scores of the medications and/or treatments and/or synergistic effectiveness scores of one or more of the medications and/or treatments. As contemplated herein, exemplary medications which may be compared and recommended using the disclosed systems and methods include, but are not limited to drug combinations containing Navitoclax—an orally active BCL2/BCL2L1 inhibitor, for estrogen receptor positive and Her2 positive breast cancer patients; AZD7762, and/or Gemcitabine, which has been shown to be effective in triple negative breast cancer

Examples of treatments which may be considered as part of the disclosed systems and methods include, but are not limited to AZD6482 (PI3K inhibitor), Linsitinib (IGF1R inhibitor), Sapitinib (inhibitor of EGFR, ERBB2 and ERBB3), Vorinostat (HDAC inhibitor), MK-1775 (also known as Adavosertib, a selective WEE1 inhibitor), or any combination of these.

In some embodiments, the systems or methods disclosed herein may comprise the step of treating a patient with one or more medications and/or treatments based on calculated individual effectiveness of the one or more medications and/or treatments, and/or based on calculated synergistic effectiveness of a combination of one or more medications and/or treatments when administered in combination.

In some embodiments, a soft k-means clustering algorithm may be used in place of creating a single cell reference atlas. As contemplated herein, a soft k-means clustering algorithm may comprise an unsupervised machine learning algorithm configured to group data into k clusters where k is some number between 1 and 100, or between 1 and 50, or between 2 and 50, or between 3 and 50, or between 5 and 25, or between 10 and 25. Unlike traditional k-means clustering algorithms, a soft k-means clustering algorithm allows one or more data points each to belong to one or more clusters, increasing the flexibility of the algorithm. As such, the mapping of the query cells also uses soft clustering for its projection into the reference atlas created by the soft k-means clustering algorithm. In some such embodiments, a k-nearest neighbor algorithm may still be used to assign labels based on the labels of the k closest points as discussed elsewhere herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of the present invention. The following working examples therefore, specifically point out the exemplary embodiments of the present invention, and are not to be construed as limiting in any way the remainder of the disclosure.

### Experiment #1=Methods

Single-cell RNA-seq datasets-A collection of publicly accessible single-cell RNA-seq count matrices was compiled from a variety of sources. When needed, gene identifiers (IDs) were translated into the current gene symbols using the reference provided by ENSEMBL BioMart for GRCh38.p13. The data were not subjected to any further normalization, transformation or quality control filters.

Drug sensitivity data-Viability data was acquired for 51 breast, 45 colorectal, and 29 pancreatic cancer cell lines in response to 2,025 clinically relevant drug combinations reported through the Genomics of Drug Sensitivity in Cancer (GDSC) cell line screening platform. The drug combinations evaluated in breast cancer cell lines were filtered, this information was used to prioritize combinations at various resolution levels according to tumor composition.

Reference atlas construction and validation—The low-dimensional embedding representing the transcriptional profile of 32 breast cancer cell lines cultured independently in ATCC recommended complete media at 37° C. and 5% CO₂ was constructed using Symphony. Symphony created an efficient and precise single-cell reference atlas through reference compression that extracts and organizes data from reference datasets into a unified and simple format that may be used to map query cells. In brief, cells are allocated in soft-cluster memberships repeatedly using Harmony, which computes weights for a linear mixture model in order to eliminate covariate-dependent effects. Symphony then compresses the reference into a mappable object, utilizing the reference-learned model parameters allowing for augmentation of the embedding with additional query cells. It maps cells into the reference without recomputing, maintaining the structure of the reference atlas. Given that all of the reference cell lines employed in this example originated from the same project (Gambardella, et al. bioRxiv (2021)), the atlas was constructed using a constant covariate, which enabled Symphony to recognize transcriptional differences across cell lines and cluster the cells appropriately. The top 50 harmonized dimensions were chosen for reference compression, and all other parameters were left unchanged.

Cross-validation, was performed, selecting a varied number of cells (from 1,000 to the size of the dataset with increasing steps of 1,000 cells) to recompute the reference and map the cells with known labels that were left out to the reference. The performance was assessed using the (accuracy) percentage of correctly classified observations divided by the total number of observations through the “caret” R package.

Single-cell RNA-seq data from other laboratories was also used and generated using a different single-cell technique to evaluate the performance of mapping three distinct breast cancer cell lines in their wild-type form, with a genetic change, and under the influence of an anticancer drug.

Transfer learning approach and validation—The transcriptional profile of 130,246 annotated single cells from 26 primary breast cancer tumors was obtained. The cells were identified as malignant based on copy number variations (CNVs), and these cells were utilized to map them into the reference embedding of breast cancer cell lines. To determine the most equivalent cell line, a k-nearest neighbors search of the closest five cells was performed following cell mapping, and the cell was assigned to the label with the highest relative frequency.

To evaluate the performance of the assignment, leave-one-out cross validation was used and the associated error for the calculated patient's tumor composition was reported in terms of breast cancer cell lines.

Activity score calculation based on patients' tumor composition-Equation 2 below was used to rank synergistic drug combinations based on the patient's tumor composition in terms of breast cancer cell lines. The activity score (A) of a two-drug (i and j) combination was computed as the scaled (between −1 and 1) average of the synergistic effect(S) of both drugs across different concentrations (d) multiplied by the proportion (P) of the cell line (l) in the tumor's composition.

$${A}_{ij} = \frac{\frac{1}{n} {\sum }_{{i}_{d} {j}_{d} = 1}^{n} ( {S}_{{i}_{d} {j}_{d}} \times {P}_{l} )}{arg max \vert \frac{1}{n} {\sum }_{{i}_{d} {j}_{d} = 1}^{n} ( {S}_{{i}_{d} {j}_{d}} \times {P}_{l} ) \vert} \qquad Equation 2$$

P-value assignation based on cell lines' response stability and patients' tumor composition—To account for the uncertainty of the cell line response to the drug combination, the authors of the dataset reported the root-mean-square error (RMSE) of the measurement. This value was used to compute an unscaled activity score (A) penalized by the RMSE of the synergistic response to the combination (Sᵢ_{_{d}}ⱼ_{_{d}}) at different concentrations (d) using Equation 3 below.

$${A}_{ij} = \frac{1}{n} {\sum }_{{i}_{d} {j}_{d} = 1}^{n} \frac{( {S}_{{i}_{d} {j}_{d}} \times {P}_{l} )}{{RMSE}_{{S}_{{i}_{d} {j}_{d}}}} \qquad Equation 3$$

This penalized score was then used to produce a P-value based on the empirical distribution after 1,000 bootstrap iterations.

Drug combination prioritization-Prioritization of drug combinations was based on the computed activity score and associated P-value accounting for the tumor composition in terms of cell lines at various degrees of resolution, ranging from a single patient to the whole cohort of patients with breast cancer. The top drug combinations identified in each scenario were labeled. The Wilcoxon rank sum test was used to compare activity scores across breast cancer molecular subgroups.

### Results

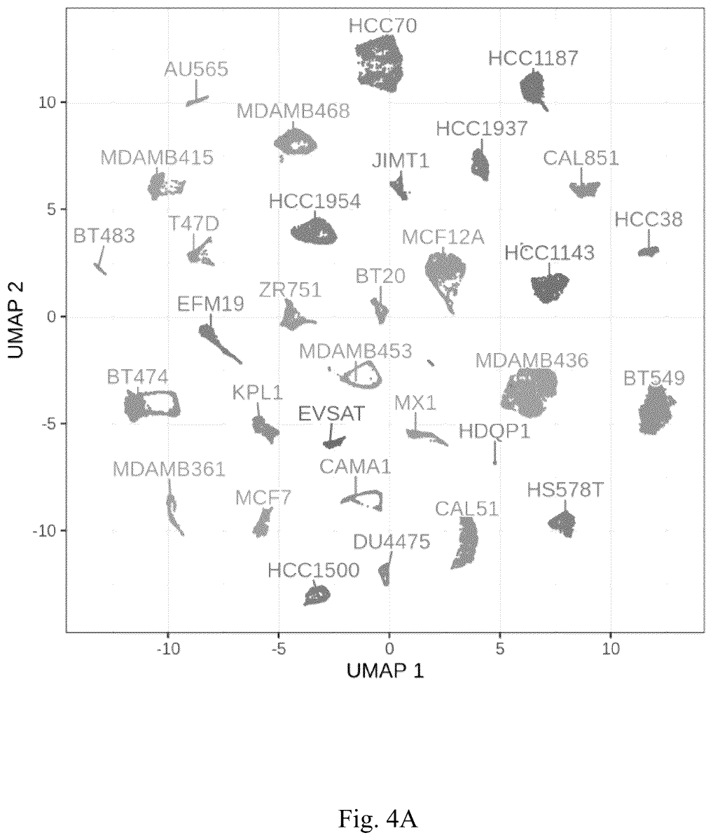

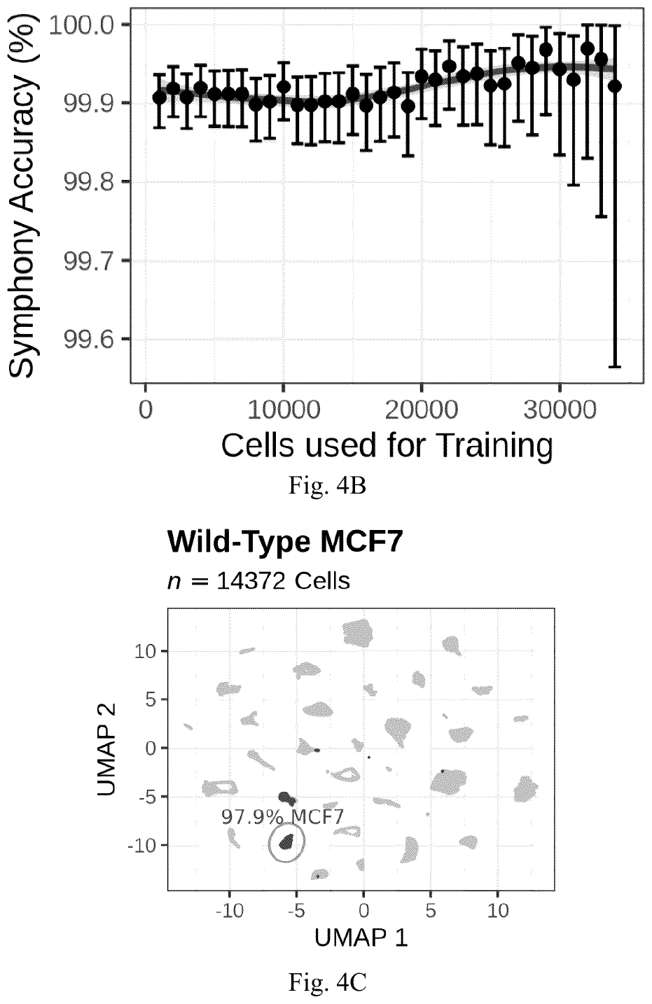

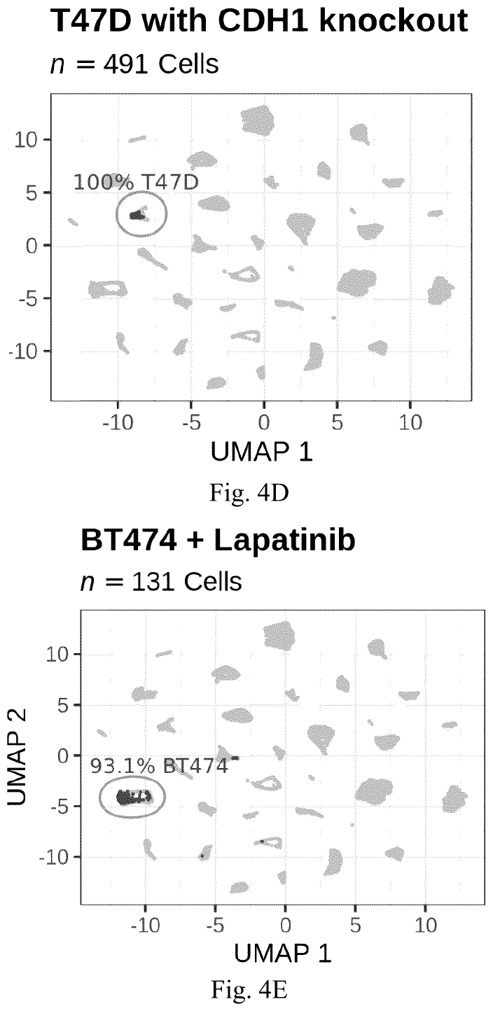

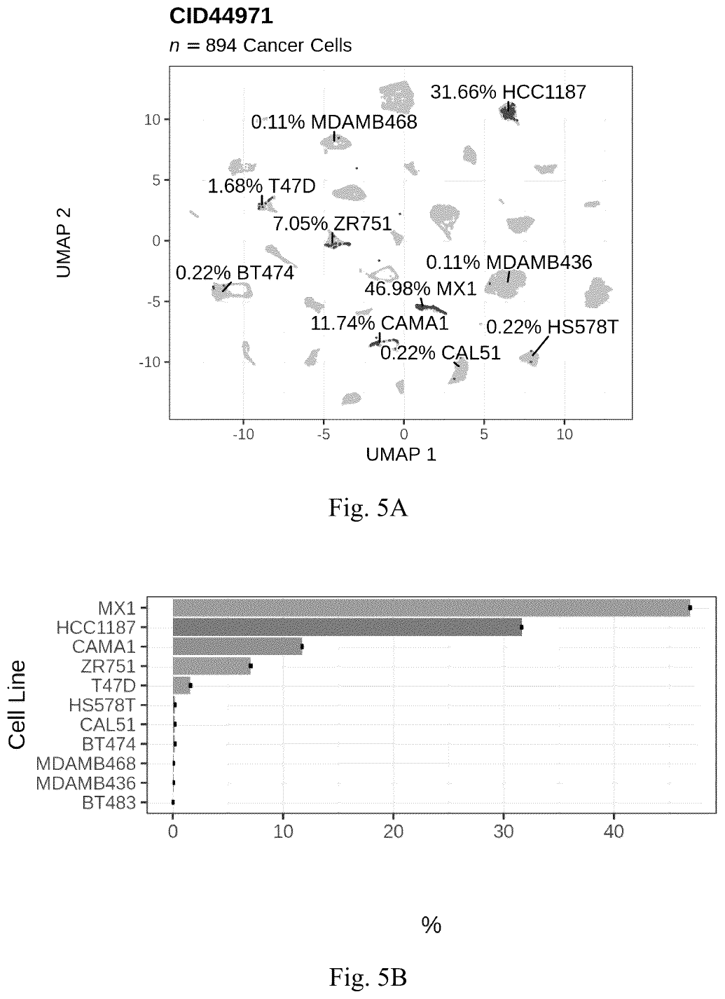

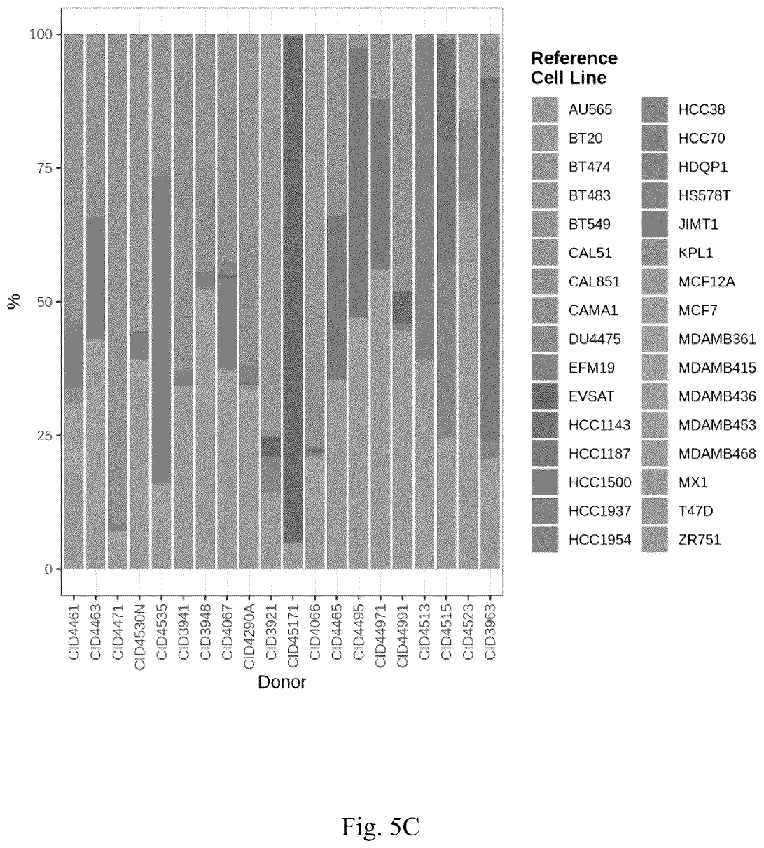

Generation of a reference breast cancer cell atlas embedding—35,276 transcriptomes were obtained from 32 ATCC-authenticated breast cancer cell lines that had been individualized using DROP-seq. These were used to generate a reference embedding as described in the methods section above (see also Macosko, et al., Cell, 2015). After compressing the transcriptional data, Symphony generated a uniform manifold approximation and projection (UMAP) using the top 50 PCA components that discriminates the 32 clusters of cells in the low-dimensional space with a minimal overlap (<1%, see FIG. 4A), which is better to differentiate cell lines than the projection reported by the dataset's original authors (G Gambardella, et al., Nature Communications, 2022; and Kang, et al., Nature communications, 2021). This indicates that the logarithm of the counts per million log(CPM+1) normalization provides a greater ability to distinguish between breast cancer cell lines than the gene frequency-inverse cell frequency (GF-ICF) normalization method.

Evaluation of the transfer learning accuracy—Two approaches were used to test the ability to distinguish between cell line transcriptomes. First, cross-validation was used to reconstruct the reference embedding of breast cancer cell lines using a randomly selected subset of cells (starting with 1,000 and increasing by 1,000 cells until the subset was the entire dataset) and then the portion of the cells with known labels that were left out was remapped to the reference embedding. Second, external transcriptomes were mapped from three different cell lines included in the breast cancer cell atlas that had been sequenced by other laboratories using a different single-cell RNA-seq technique to the reference embedding generated using all 35,276 cells. In both cases, after remapping, the labels for the cells were assigned using a k-nearest neighbors search of the closest five cells and selecting the label with the highest relative frequency.

The cross-validation approach's results support the use of this technique in clinical settings. Across all cases, the correct cell line label was recovered with greater than 99.5 percent accuracy (Binomial test, P<0.01 in all cases, FIG. 4B). However, these results may be distorted due to the fact that all transcriptomes used to generate the reference embedding and to assess performance were sequenced in the same batch. To avoid bias, the performance of mapping transcriptomes from three distinct breast cancer cell lines included in the reference embedding were evaluated in their wild-type state, with a genetic mutation, and under the influence of an anticancer drug sequenced using a different single-cell RNA-seq technique and by other laboratories.

To begin, 14,372 single-cell transcriptomes sequenced using the 10× Chromium method from the wild-type MCF7 cell line were mapped into the reference embedding of breast cancer cell lines to assess the efficacy of mapping transcriptomes from a different single-cell RNA-seq approach. It was found that 14,073 (97.92%) of them were recognized and labeled correctly as MCF7 (FIG. 4C). The remaining cells were predominantly labeled as KPL1 (278 cells, 1.93%), and ZR751 (16 cells, 0.11%), two other estrogen receptor positive luminal cell lines linked to the same molecular subtype of breast cancer as MCF7. Furthermore, contamination of the KPL1 cell line by an MCF7 derivative has been documented in prior work. If this is the case for the parental cell line used by the dataset's authors, then the cell label assignment may be considered as 99.85% accurate.

With reference to FIG. 4A-FIG. 4E, graphical data is shown related to transfer-learning accuracy evaluation. FIG. 4A shows a Uniform Manifold Approximation and Projection (UMAP) displaying the harmonized transcriptional profile of 32 breast cancer cell lines sequenced using DROP-seq. FIG. 4B shows an accuracy evaluation using cross-validation. The percentage of cells utilized for training is displayed, as well as the accuracy and 95% confidence intervals computed using the binomial test. Accuracy is defined as the percentage of correctly classified observations divided by the total number of observations.

FIG. 4C shows an accuracy evaluation using wild-type cells from a different single-cell technology. Transcriptomes of cells derived from the wild-type MCF7 cell line that were sequenced using 10× Chromium were appropriately labeled in 97.9% of cases. FIG. 4D shows an accuracy evaluation using cells from a different single-cell technology carrying a genetic modification. Transcriptomes of cells originating from the T47D cell line carrying the CDH1 gene knockout through CRISPR and sequenced using 10× Chromium were labeled correctly in all cases. FIG. 4E shows an accuracy evaluation using cells from a different single-cell technology under cancer treatment. Transcriptomes of cells obtained from the BT474 cell line that were treated with 1 μM of Lapatinib for 10 days and sequenced using 10× Chromium were identified correctly in 93.1% of instances.

Because cancer cells have a high mutational load, the impact of carrying a genetic mutation related with epithelial cell phenotypic identity was investigated throughout the mapping process. 491 cell transcriptomes obtained from the T47D cell line bearing CDH1 deletion created using CRISPR techniques were mapped for this purpose. It was found that all 491 (100%) cells were correctly assigned to the T47D cluster in the reference embedding (FIG. 4D), showing that genetic alterations, even those linked with phenotypic identity in breast cancer cells, seem to have little effect on the accuracy of the disclosed classifier.

Additionally, changes in anticancer drug response constitute a significant source of variability in cancer patients' cell transcriptomes. As a result, 131 transcriptomes obtained from the BT474 cell line after ten days of treatment with 1 μM of Lapatinib were mapped. This was done to determine the influence of anticancer drugs on the cell identification during the mapping procedure. It was found that 122 (93.13%) of the cells accurately mapped to the BT474 cluster (FIG. 4E) in the reference embedding. The remaining cells were attributed to ZR751 (8 cells, 6.11%) and CAMA1 (1 cell, 0.76%), two metastatic estrogen receptor-positive luminal cells that are not of the same molecular subtype (H)r2⁺) as BT474. This finding provides evidence that anticancer drugs affect the identity of cellular transcriptomes, and therefore slightly reduce the ability to map treated cells' transcriptomes into the reference embedding of breast cancer cell lines.

In general, when applied to transcriptomes derived from the same cell lines, the mapping approach and transfer learning procedure provide reliable results with accuracies above 90% in all cases, even when they were characterized using a different single-cell RNA-seq technique, affected by a genetic mutation, or undergoing treatment with an anticancer drug. This evidence supports its usage in clinical practice for classifying patients' malignant tumor cells based on their most comparable cell line.

Application of transfer learning to patients' tumor cells—Transcriptomes of 30,246 single cells were obtained from 11 ER⁺, 5 HER2⁺, and 10 TNBC primary tumors and the cells designated as malignant by the dataset's authors were selected using CNV characterization. After filtering the non-cancerous cells, the transcriptomes for 24,489 cells from 20 tumors (9 ER⁺, 3 HER2⁺, and 8 TNBC) were kept. The transcriptomes of each patient's associated malignant cells were used to map them into the reference embedding atlas established using breast cancer cell lines.

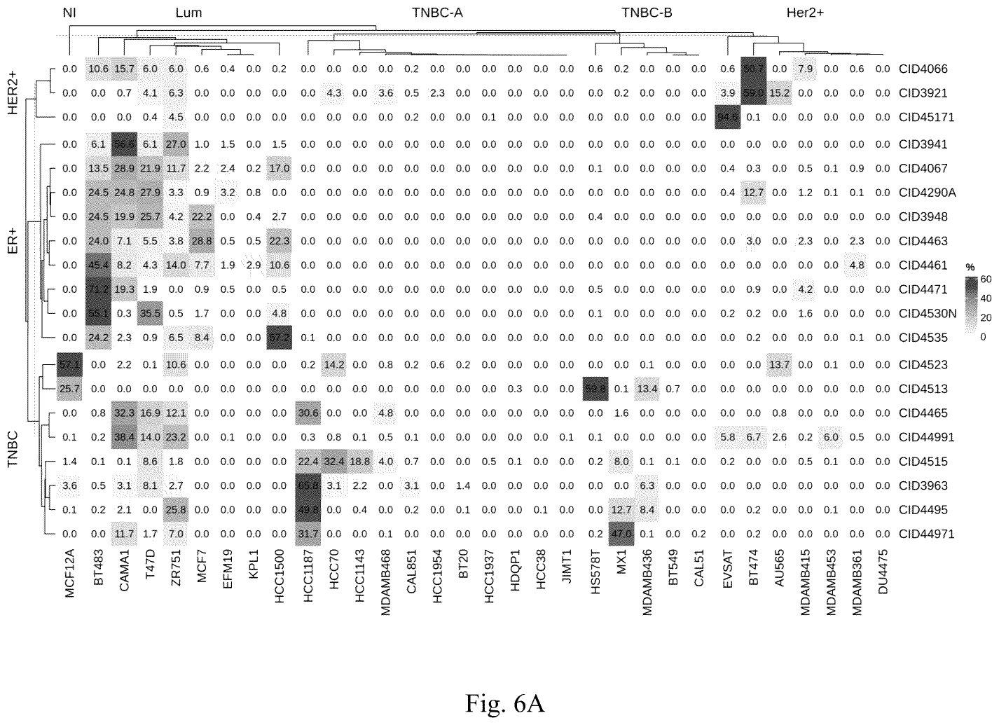

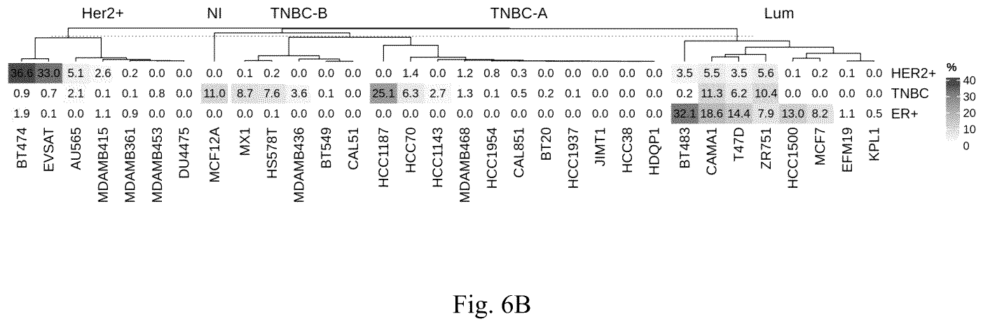

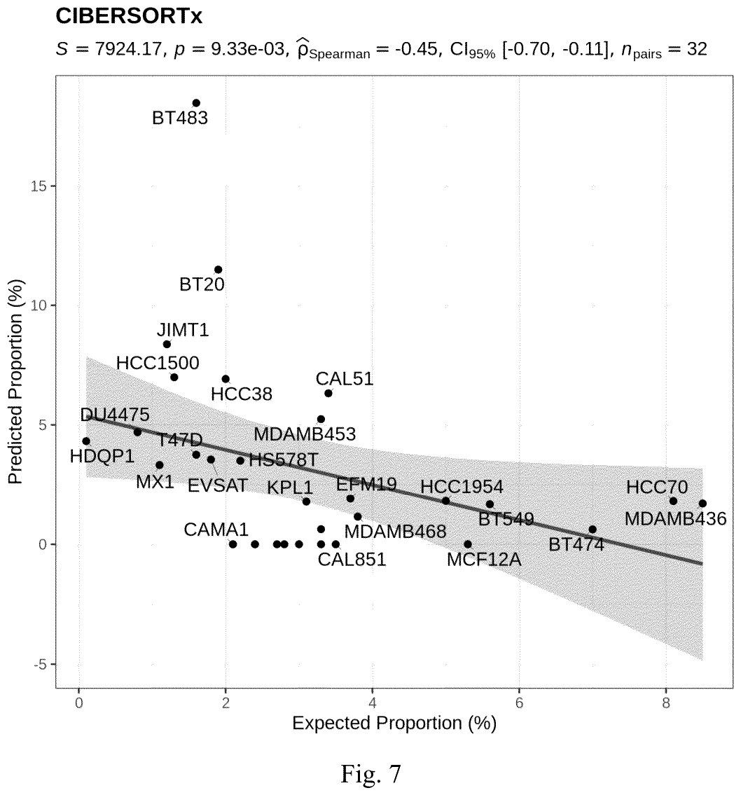

The 894 cancer cells reported for donor CID44971 are discussed here to serve as an example of the process. When the cell transcriptomes were mapped into the reference embedding, it was found that 420 cells (46.98%) were mapped to MX1, 283 (31.66%) to HCC1187, 105 (11.74%) to CAMA1, 63 (7.05%) to ZR751, 15 (1.68%) to T47D, 2 (0.22%) to BT474, CAL51, and HS578T, and 1 (0.11%) to MDAMB436 and MDAMB468 (FIG. 5A). This specific distribution across cell lines is unlikely to happen by chance (χ² test, P<1×10⁻⁶).

Metadata associated with the dataset revealed that CID44971 cells were from a female donor affected with a breast cancer tumor of the TNBC molecular subtype. Confirming that diagnosis and the accuracy of the mapping and transfer learning approach, 709 (79.31%) of the cells' transcriptomes were mapped to cell lines representing the TNBC molecular subtype, the remaining 185 cells were identified as derivates of luminal (183 cells, 20.47%) and HER2⁺ (2 cells, 0.22%) cell lines. A leave-one-out cross validation analysis was performed to validate the stability of the cell line assignation for the transcriptomes derived from the CID44971 donor (FIG. 5B). The cross validated mean proportions were found to be highly correlated with those identified initially (ρ=0.97, Spearman's rank correlation P=5.5×10⁻²⁰) with small RMSE (0.02).

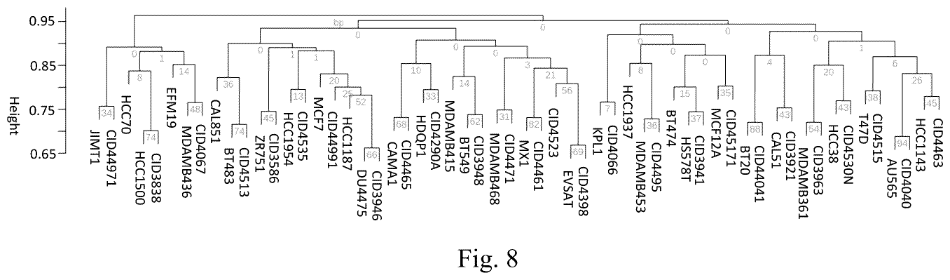

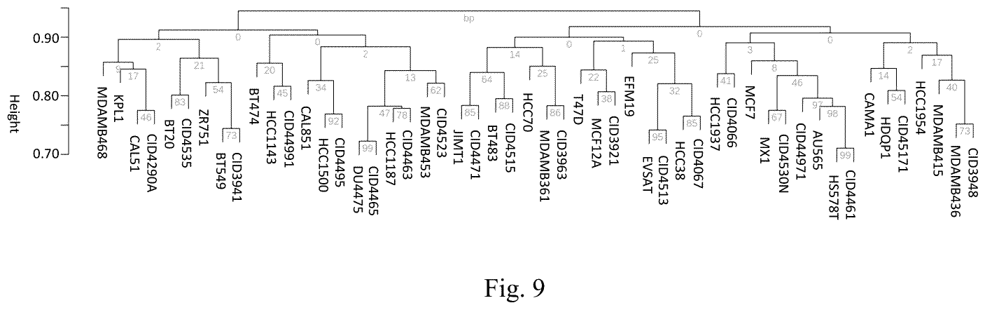

The computed cancerous cell proportions in terms of cell lines for all 20 donors are displayed in FIG. 5C and numerically in FIG. 6A. As previously described, cancer tumor cells are highly heterogeneous, facilitating the development of resistance to cancer therapies. The observed interpatient and intratumor heterogeneity, as seen in this patient cohort, makes drug development and clinical trial design difficult. Identifying the most common cell lines found in patients' tumors across different molecular subtypes may aid in resource allocation and the development of treatments that will benefit a large proportion of the patient population (see Garrido-Castro, et al., Cancer discovery, 2019).

Prioritizing cell lines for breast-cancer drug development and testing—The relative frequencies identified after computing the cancerous cell proportions were averaged in terms of cell lines for all 20 donors to identify the most common cell lines found in patients' tumors by molecular subtype. The cell lines in which the cells from patients' tumors were mapped were found to be enriched with those developed as surrogates for each molecular subtype, confirming again the accuracy of the mapping and transfer learning approach. Luminal cell lines were shown to be abundant in estrogen receptor positive tumors, accounting for 95.79% of malignant cells (χ² test, P=8.46×10⁻⁸⁷). It was also observed that 77.46% of cells in HER2⁺ tumors mapped to cell lines designed to represent this category (χ² test, P=1.02×10⁻¹³⁰), but only 56.22% of cells in TNBC mapped to cell lines representing this molecular subtype of breast cancer (χ² test, P=3.29×10⁻⁴⁴). TNBC tumors were also found to be infiltrated with a modest proportion of luminal cell lines (28.07%) and a small proportion of HER2⁺ cell lines (4.72%).

Current breast cancer research relies on a small number of cell lines, with MCF7, T47D, and MDAMB231 accounting for more than two-thirds of all cell lines used in studies, raising the question of how representative these few cell lines are of the diversity of breast tumors with distinct clinical characteristics. This level of resolution, which enables classification of patients' tumor cells into cell lines, is relatively recent and the result of enormous advances in both experimental and computational biology (Vamathevan, et al., Nature reviews Drug discovery, 2019). The present disclosure serves to characterize the predicted subpopulations of cell lines found in patients' tumors. In contrast to conventional wisdom, it was found that BT483 and CAMA1 are the most frequently occurring cells in estrogen receptor positive tumors, accounting for more than 50% of cells. For HER2⁺ tumors, BT474 and EVSAT are the two most prevalent, accounting for roughly 70% of tumor cells. The high frequency of BT474 in HER2⁺ tumors has previously been reported (Oren, et al., Nature, 2021), lending additional support to the mapping and transfer learning approach's high accuracy. In the case of TNBC tumors, no pair of cell lines was found that accounted for the vast majority of the tumor's cells. HCC1187 and MX1 are the two most common cell lines, accounting only for 33.8 percent of tumor cells (see FIG. 6B).

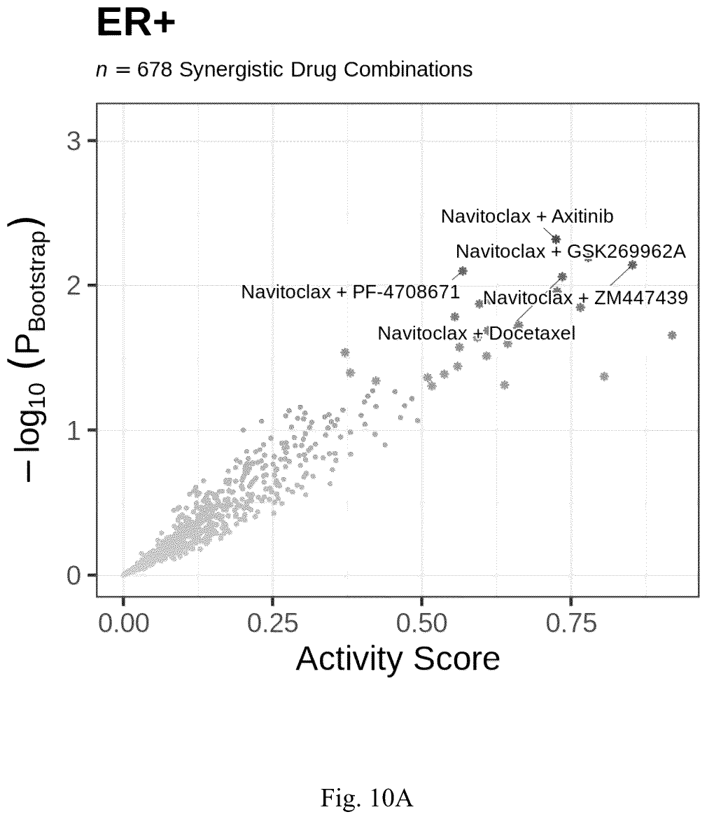

A pseudo bulk sample was constructed by randomly selecting 1000 transcriptomes from the pool transcriptomes available for the 32 breast cancer cell lines. As a reference, the sum of all UMIs for all cells in each cell line was used. Following that, both the constructed sample and the reference were normalized using the logarithm of the counts per million. CIBERSORTx was used to deconvolve the constructed sample and recover the fraction of cell lines whose true proportion was known. The result is a negative correlation (p=−0.45, Spearman's rank correlation, P=9.33×10⁻³) between the predicted and known cell composition (see FIG. 7). Bisque was also evaluated, but Bisque requires paired bulk and single cell RNA-seq data for a portion of the population in order to generate a reference expression profile and learn gene-specific bulk expression transformations for robust RNA-seq data decomposition. Due to the lack of such pairing profiles, their application to the TCGA data was not feasible.

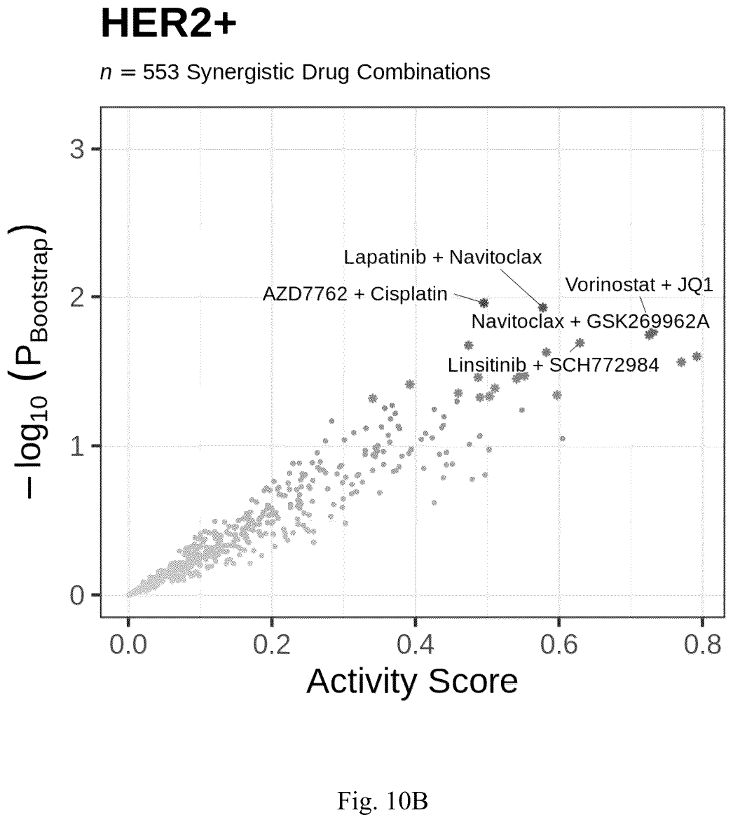

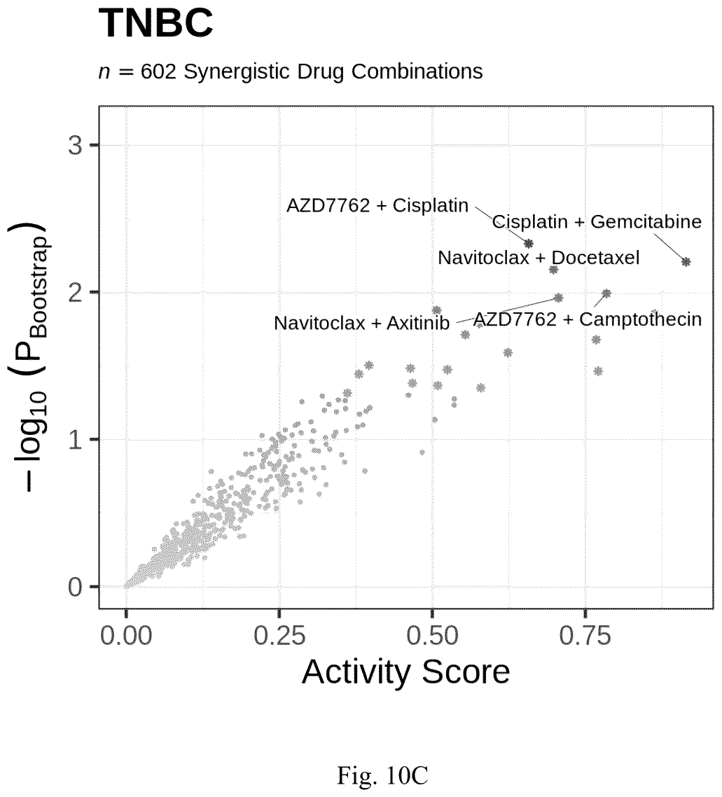

Driven by the fact that deconvolution did not produce useful results, Spearman's correlation was used to determine whether clustering was helpful in determining at least the most frequent cell line making part of the tumor samples. To accomplish this, the pseudo-bulk profile was computed, which includes all cells in the tumor (for reference in cases where FACS is not possible) and only cancerous tumor cells. The generated pseudo-bulk profiles were normalized using the logarithm of the counts per million and then combined with the cell line pseudo-bulk profiles. The normalized pseudo-bulk profiles of samples and cell lines were clustered, and the association's uncertainty was evaluated using bootstrap through “pvclust” (see Suzuki et al., Bioinformatics, 2006). In both cases, whether all cells within the tumor sample (see FIG. 8) or only cancerous cells (see FIG. 9), were considered, the Spearman's correlation failed to recognize the most frequently occurring cell line within the tumor samples.

Prioritizing synergistic drug combinations for personalized medicine and clinical trials-296,707 survival statistics were obtained from those generated from the testing of 2,025 clinically relevant two-drug combinations in 51 breast, 45 colorectal, and 29 pancreatic cancer cell lines (Jaaks, et al., Nature, 2022). After filtering out the ones from breast cancer cell lines, 156,065 records remained representing the effect of 1,275 two-drug combinations at different concentrations.

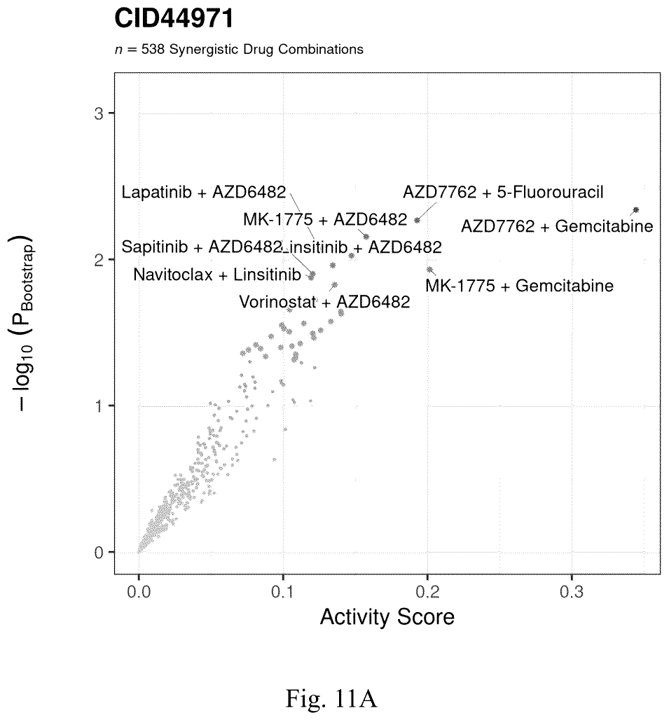

To evaluate the proposed activity score performance, it was computed using a uniform proportion for all cell lines representing each breast cancer subtype (see FIG. 10A, FIG. 10B, and FIG. 10C). It was found that the combination of Cisplatin and Gemcitabine was recommended as the best candidate for TNBC patients under this setting, confirming its accuracy. Cisplatin and Gemcitabine are used as first-line therapy in patients affected with metastatic triple negative breast cancer.

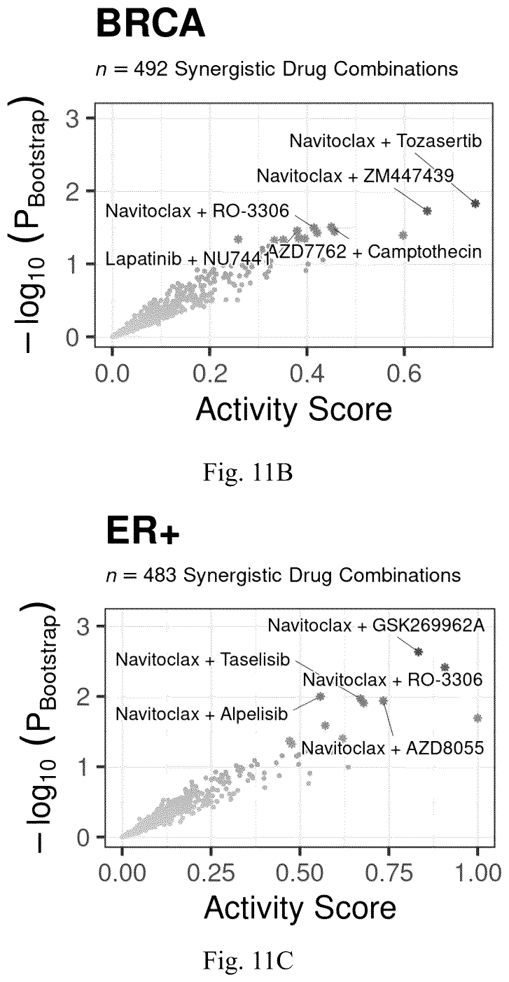

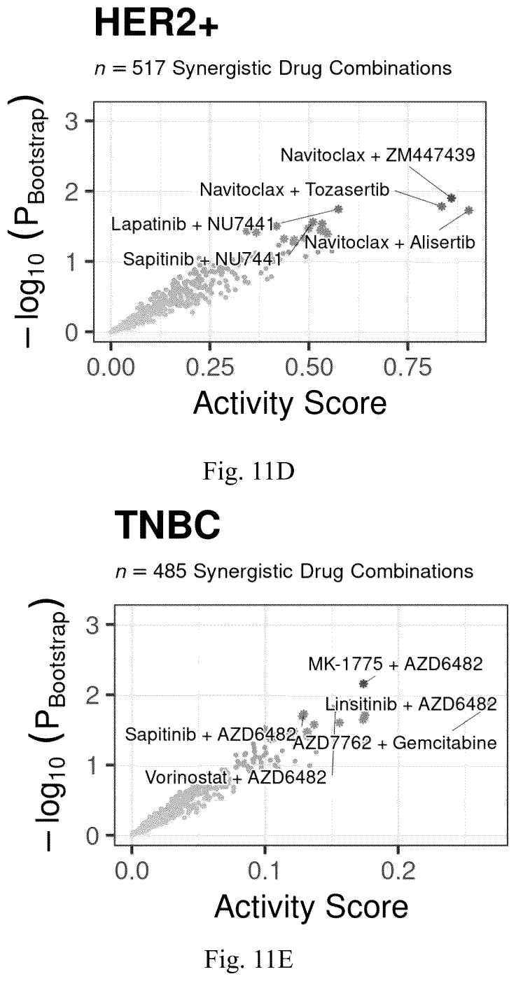

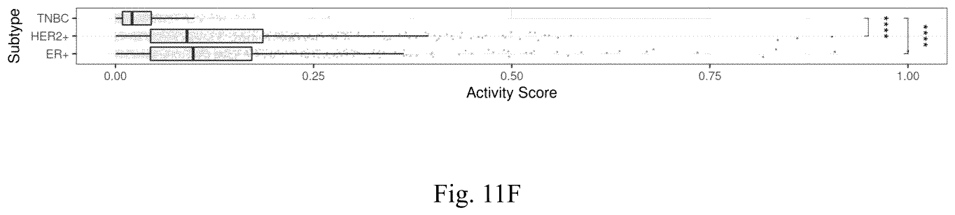

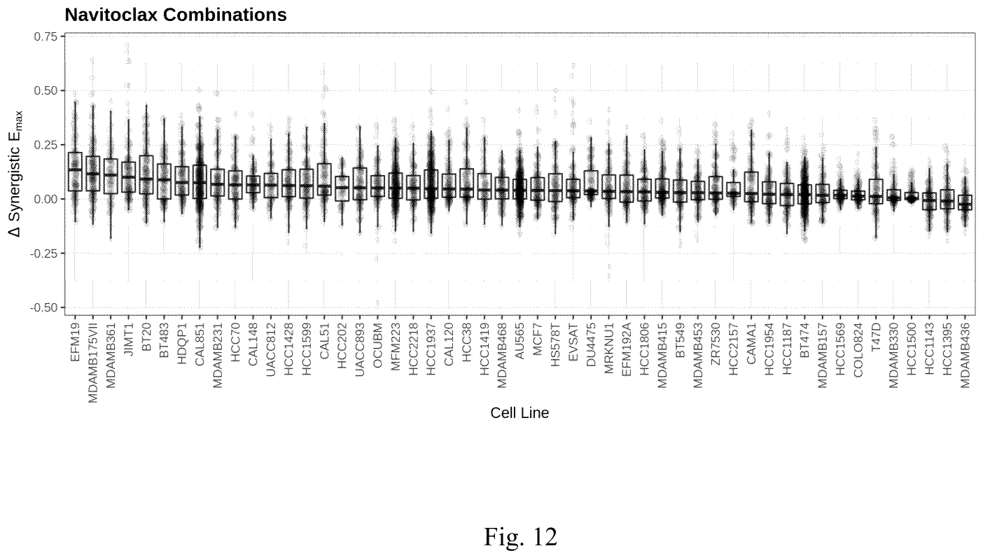

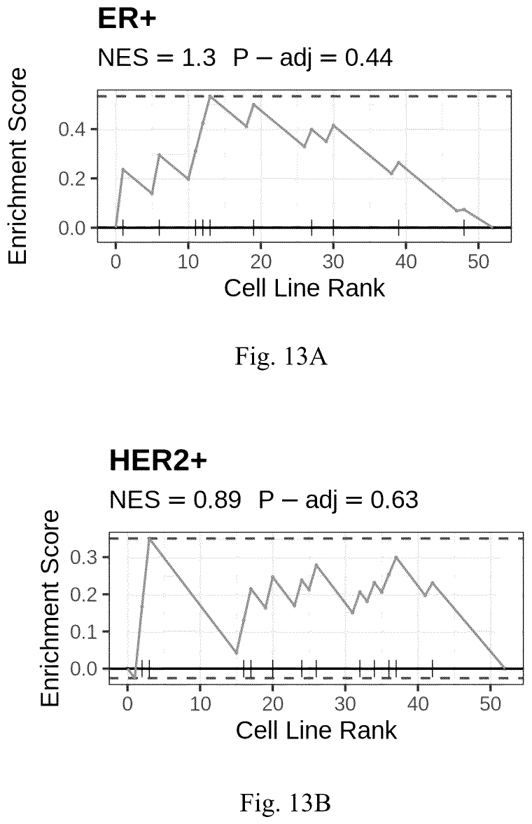

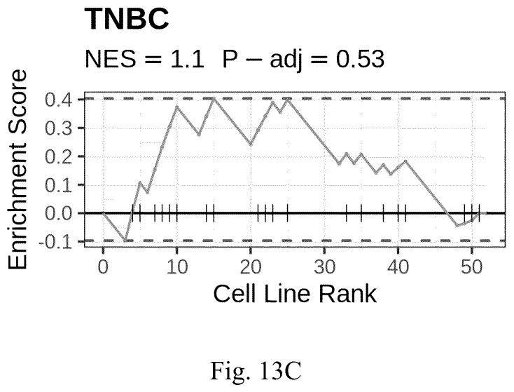

Once its performance was confirmed, the activity score was computed for each patient and molecular subtype of breast cancer, as well as for the entire cohort, as described in Equation 2 above, using the identified proportions for each cell type in each case. As expected, given the diversity of tumor compositions, the top candidates for each molecular subtype and patient were different (see FIG. 11A, FIG. 11B, FIG. 11C, FIG. 11D, and FIG. 11F) Consistent with what was reported by the authors of the dataset, drug combinations including Navitoclax,—an experimental orally active BCL2, BCL2L1, and BCL2L2 inhibitor—are enriched among the top candidates displaying synergistic effects for tumors from the entire cohort of patients (Hypergeometric test, P=5.30×10⁻⁵) and for the estrogen receptor positive (Hypergeometric test, P=9.53×10⁻⁷) and HER2⁺ (Hypergeometric test, P=5.30×10⁻⁵) molecular subtypes of breast cancer (FIG. 11B, FIG. 11C, FIG. 11D), but not for TNBC tumors (FIG. 11E). This result is directly related to the unique tumor compositions found, rather than a generalized response of TNBC cell lines to Navitoclax combinations (see FIG. 12, FIG. 13A, FIG. 13B, and FIG. 13C).

It was found that the best candidate for TNBC malignancies was the combination of AZD7762 and Gemcitabine, which has been shown to be effective in triple negative breast cancer. However, development of AZD7762 has been suspended due to significant heart side effects. An enrichment of drug combinations including AZD6482 was also identified (Hypergeometric test, P=1.9×10⁻⁹). AZD6482 is an ATP competitive inhibitor of the phosphatidylinositol 3-kinase (PI3K) p110β isoform with an IC₅₀ of 0.01 nM. PI3K is an important target for breast cancer, because PTEN loss (the sixth most common mutation in breast cancer patients' tumors) causes upregulation of PI3K|AKT, through the p110β isoform. When AZD6482 is combined with Linsitinib (selective inhibitor of IGF1R), Sapitinib (a reversible ATP competitive inhibitor of EGFR, ERBB2, and ERBB3), Vorinostat (an HDAC inhibitor) or with MK-1775 (also known as Adavosertib, a selective WEE1 inhibitor), it displays synergistic effects against breast cancer cell lines. These findings support AZD6482's versatility for the treatment of triple negative breast cancer and provide options for managing resistance development via four distinct pathways.

When all of the computed activity scores were compared across molecular subtypes of breast cancer, it was discovered that those computed for TNBC were significantly lower (FIG. 11F) than those computed for HER2⁺ (ANOVA, P=2.27×10⁻⁴) and ER⁺ subtypes (ANOVA, P=1×10⁻⁷). Finding effective treatments for TNBC has been a difficult task; here, evidence is provided that such difficulty is highly associated with the hyper-heterogeneity of the cancerous cells that comprise TNBC tumors. Finding a drug or a combination that is effective among two top cell lines that account for the majority of tumor cells is relatively simple, but finding one that is effective across many cell lines with different molecular phenotypes is extremely challenging. Furthermore, having a proportion of cells with a different molecular phenotypes that do not respond to treatment is a good source of drug resistance development.

### DISCUSSION

Evidence is presented herein that supports the use of transfer learning in clinical practice for the contextualization of breast cancer patient tumor cells. It was demonstrated that this procedure remains stable even when cells' transcriptomes are characterized using a different experimental technique or when cells have a genetic mutation. The efficacy of this method was tested on a small group of breast cancer patients. The findings add to the understanding of the composition and characteristics of breast cancer tumors across multiple molecular subtypes. The most synergistic drug combinations for prioritization were predicted based on the identified compositions of the tumors. In this case, predictions were made based on patient characterizations from a small (n=20) cohort.

Drug development and testing is a lengthy process that begins with testing in cell lines that mimic the phenotypic and genetic variability of patients. The disclosed approach will hasten the process of developing truly personalized (n=1) pharmacological options for patients. Although the entire dataset of cellular responses to drug combinations was in this study, the authors of the dataset carefully labeled them based on the approval level for each compound. This allowed for the selection of drugs that were already defined as safe and approved, for their use in patients with limited treatment options.

### Experiment #2

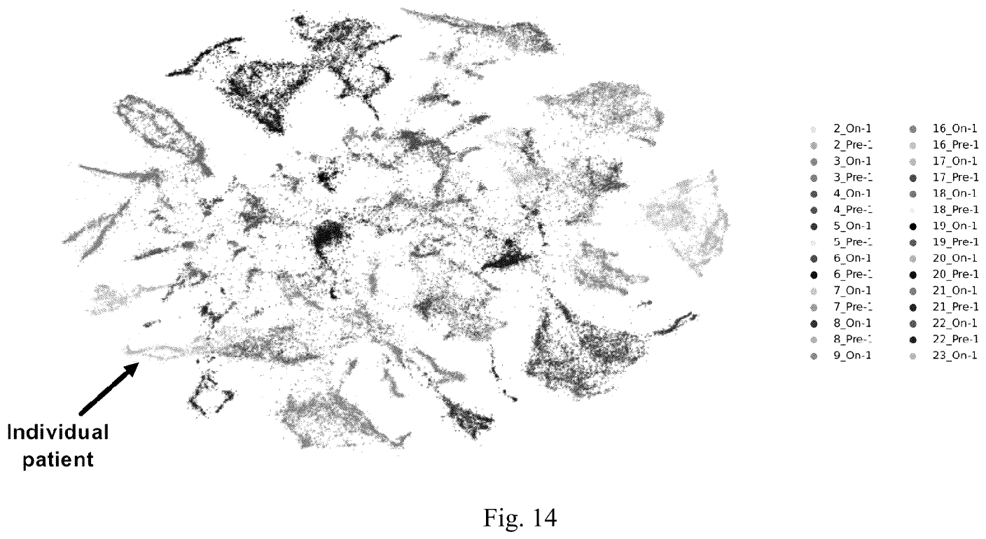

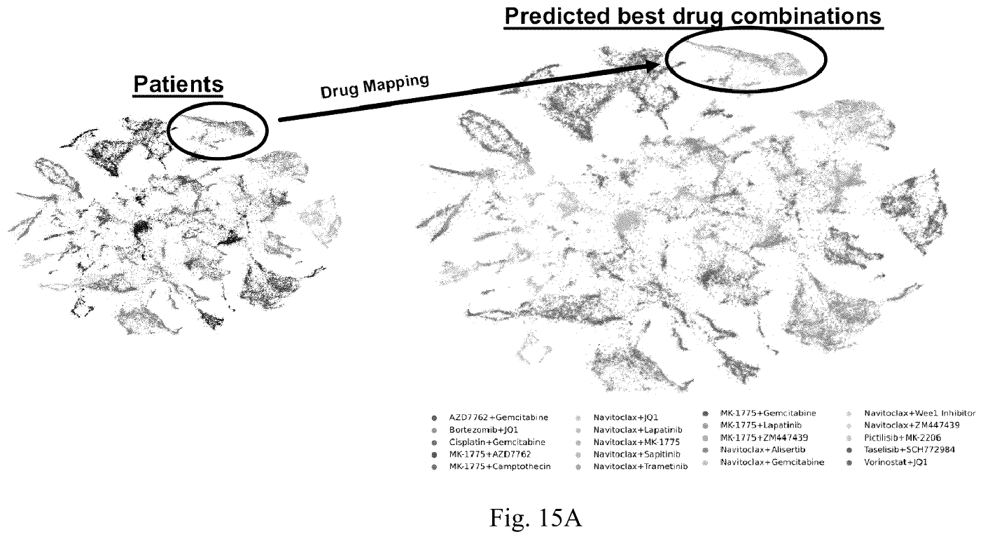

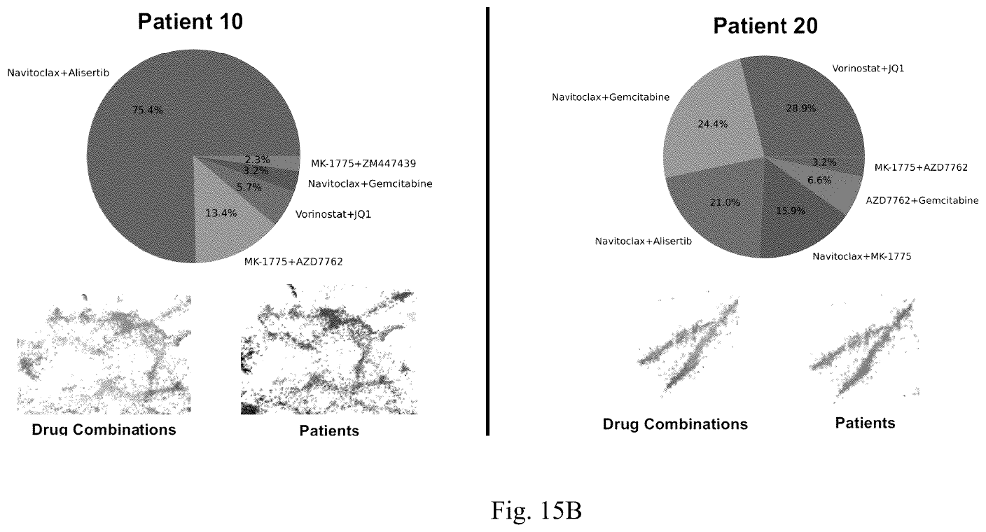

Soft k-means clustering, as disclosed herein, was used to map 30 patient single cell RNA-seq data, and the data was clustered into distinct subcommunities in UMAP (see FIG. 14), where distinct colors represent distinct patient samples. In contrast to the previous methods disclosed in Experiment #1, the new version of the disclosed method is capable of predicting personalized drug combinations for 100% of the patients (see FIG. 15A and FIG. 15B), with no limitation observed yet.

The new version of the method appears to exhibit better performance by offering more unique drug options for distinct patients, in comparison to the previous version where most cancer patients of the same subtype (e.g., HER2+) were prescribed the same drug or combination of drugs. The new method offers more unique, individualized drug combination options, which provides more benefits for all patients, especially if they are at an advanced stage of cancer progression.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating a subject for a disease, comprising:
obtaining a first data set of cell transcriptomic data related to samples of cell lines taken from subjects diagnosed with the disease;
obtaining a second set of cell transcriptomic data related to samples of cell lines taken from a subject to be treated;
obtaining a set of effectiveness data related to a plurality of candidate medications or treatments related to the samples;
calculating a lower-dimensional model from the first set of cell transcriptomic data in order to group the cell lines into a plurality of groups;
assigning the second set of cell transcriptomic data to at least one group of the plurality of groups;
calculating a set of candidate medication or treatment combinations from the plurality of candidate medications or treatments, each combination in the set comprising N treatments or medications, where N>1;
calculating a synergistic effectiveness of each combination in the set of candidate medication or treatment combinations related to each group of the plurality of groups of cell lines;
selecting a treatment combination from the set of candidate medication or treatment combinations whose synergistic effectiveness is above a threshold for the at least one group; and
treating the subject with the selected treatment combination.

2. The method of claim 1, wherein the step of calculating the lower-dimensional model from the first set of cell transcriptomic data comprises the step of discarding one or more dimensions or parameters of the data.

3. The method of claim 1, wherein the step of calculating the lower-dimensional model from the first set of cell transcriptomic data comprises the step of combining two or more dimensions or parameters of the data.

4. The method of claim 1, wherein the step of calculating the lower-dimensional model from the first set of cell transcriptomic data comprises the use of a dimensionality reduction technique selected from principal component analysis and grouping high-dimensional data into clusters.

5. The method of claim 1, wherein the step of calculating the lower-dimensional model from the first set of cell transcriptomic data comprises assigning weights to one or more parameters of the first set of cell transcriptomic data and discarding parameters weighted below a threshold.

6. The method of claim 1, wherein the step of calculating the lower-dimensional model from the first set of cell transcriptomic data comprises assigning weights to one or more parameters of the first set of cell transcriptomic data and selecting the N highest weighted parameters.

7. The method of claim 1, wherein the step of calculating the lower-dimensional model from the first set of cell transcriptomic data comprises using k-means clustering.

8. The method of claim 1, wherein the step of assigning the second set of cell transcriptomic data to at least one group of the plurality of groups comprises calculating the k-nearest neighbor of each element of the second set of cell transcriptomic data.

9. The method of claim 1, wherein the disease is cancer and wherein the first set of cell transcriptomic data comprises cells selected from cancer cell lines, tumor cell lines, breast cancer cell lines, pancreatic cancer cell lines, or other tumor cell lines.

10. A system for selecting a treatment combination for a subject, comprising a non-transitory computer-readable medium with instructions stored thereon, which when executed by a processor, perform steps comprising:
obtaining a first data set of cell transcriptomic data related to samples of cell lines taken from subjects diagnosed with the disease;
obtaining a second set of cell transcriptomic data related to samples of cell lines taken from a subject to be treated;
obtaining a set of effectiveness data related to a plurality of candidate medications or treatments related to the samples;
calculating a lower-dimensional model from the first set of cell transcriptomic data in order to group the cell lines into a plurality of groups;
assigning the second set of cell transcriptomic data to at least one group of the plurality of groups;
calculating a set of candidate medication or treatment combinations from the plurality of candidate medications or treatments, each combination in the set comprising N treatments or medications, where N>1;
calculating a synergistic effectiveness of each combination in the set of candidate medication or treatment combinations related to each group of the plurality of groups of cell lines; and
selecting a treatment combination from the set of candidate medication or treatment combinations whose synergistic effectiveness is above a threshold for the at least one group.

11. The system of claim 10, wherein the step of calculating the lower-dimensional model from the first set of cell transcriptomic data comprises the step of discarding one or more dimensions or parameters of the data.

12. The method of claim 10, wherein the step of calculating the lower-dimensional model from the first set of cell transcriptomic data comprises the step of combining two or more dimensions or parameters of the data.

13. The method of claim 10, wherein the step of calculating the lower-dimensional model from the first set of cell transcriptomic data comprises the use of a dimensionality reduction technique selected from principal component analysis and grouping high-dimensional data into clusters.

14. The method of claim 10, wherein the step of calculating the lower-dimensional model from the first set of cell transcriptomic data comprises assigning weights to one or more parameters of the first set of cell transcriptomic data and discarding parameters weighted below a threshold.

15. The method of claim 10, wherein the step of calculating the lower-dimensional model from the first set of cell transcriptomic data comprises assigning weights to one or more parameters of the first set of cell transcriptomic data and selecting the N highest weighted parameters.

16. The method of claim 10, wherein the step of calculating the lower-dimensional model from the first set of cell transcriptomic data comprises using k-means clustering.

17. The method of claim 10, wherein the step of assigning the second set of cell transcriptomic data to at least one group of the plurality of groups comprises calculating the k-nearest neighbor of each element of the second set of cell transcriptomic data.

18. The method of claim 10, wherein the disease is cancer and wherein the first set of cell transcriptomic data comprises cells selected from cancer cell lines, tumor cell lines, breast cancer cell lines, pancreatic cancer cell lines, or other tumor cell lines.
